SEQUENCE LISTING (12 sequences)

SEQ ID NO: 1 - DNA, 2667 bp, Homo sapiens
gttttgttaggcaaaaagagactattgaaagctgagactttagaattgagtgacttatatgttagtgataagaagaaggatatgtctccaccctttatttgtgaggagacagatgaacaaaagcttcaaactctagacatcggtagtaacttggagaaagaaaaattagagaattccagatccttagaatgcagatcagatccagaatctcctatcaaaaaaacaagtttatctcctacttctaaacttggatactcatatagtagagatctagaccttgctaagaaaaaacatgcttccctgaggcagacggagtctgatccagatgctgatagaaccactttaaatcatgcagatcattcatcaaaaatagtccagcatcgattgttatctagacaagaagaacttaaggaaagagcaagagttctgcttgagcaagcaagaagagatgcagccttaaaggcggggaataagcacaataccaacacagccaccccattctgcaacaggcagctaagtgatcagcaagatgaagagcgacgtcggcagctgagagagagagctcgtcagctaatagcagaagctcgatctggagtgaagatgtcagaacttcccagctatggtgaaatggctgcagaaaagttgaaagaaaggtcaaaggcatctggagatgaaaatgataatattgagatagatactaacgaggagatccctgaaggctttgttgtaggaggtggagatgaacttactaacttagaaaatgaccttgatactcccgaacaaaacagtaagttggtggacttgaagctgaagaagctcctagaagttcagccacaggtggcaaattcaccctccagtgctgcccagaaagctgtaactgagagctcagagcaggacatgaaaagtggcacagaagatctccggactgaacgattacaaaaaacaacagaacgttttagaaatcctgttgtgttcagcaaagattctacagtcagaaaaactcaacttcagtctttcagccaatatattgagaatagaccagagatgaaaaggcagagatcaatacaggaagatacaaagaaaggaaatgaggagaaggcagcgataactgaaactcagaggaagccatcagaagatgaagtgcttaataaagggttcaaagacaccagtcagtatgtagtaggagaattggcagcactagagaatgagcaaaagcaaattgacacccgtgccgcgctggtggagaagcgccttcgctatctcatggacacaggaaggaacacagaagaagaagaagctatgatgcaggaatggtttatgttagttaataagaaaaatgccttaataaggagaatgaatcagctctctcttctggaaaaagaacatgatttagaacgacggtatgagctgctgaaccgggaattgagggcaatgctagccattgaagactggcagaagaccgaggcccagaagcgacgcgaacagcttctgctagatgagctggtggccctggtgaacaagcgcgatgcgctcgtcagggacctggacgcgcaggagaagcaggccgaagaagaagatgagcatttggagcgaactctggagcaaaacaaaggcaagatggccaagaaagaggagaaatgtgttcttcagtagccatcagatcagaaagaatctctcccaacattttagagtcttgcttcccaaaccagaaaaagtcagactcattgttgatttaaaacttttaacattttgtttggctggattgtactactttacctctactttaccaccaccacccttttcctccctcctttccaaataatatacagaactccaaaatagcttcatttaaggatttttttgtgagttaacaatttccttgaaatcctgtgaaatagatttgcacagacaccttgtgagtgattggtattggaggtgttcaagaaactgttcgaaaaagaacaaaaacacttccctcgttattttctctcattttttgatgagaggaaaatttgaaacattattcttgttgttgttggtaatagcataatgacagtgggaggggtacaaggggataagaaaaatgtcatgatttttttccggtcctgccacatgtaacacttactctgttacctaaattttatagttagatcatatccaatctacttattaaactgtgttctatttaccagtggagtttttctgcagtggttgcgtttcactgtaaggataatggagttcctctcctctgctttcctcagaggatggtcctttaacatagccagaaacaagccctgtggtttgaaggtgagctgtgaggatgggactaattgatatgcaccagtttacaaagacagtcttatcatccgagaatacaccatctttttctctggataattatttcttacatcatgcttgattcctacattttgttgggtttcaacattggctcacgaatgctgttaatatttattctgtattgataaaaagtctgtcttgccactacaagtaaatcccccatttaatattttcttctttagcatagcactgtcattttttgtgaaaatggttatgtttatttattacaatactgagtcatatataaattttcaataaaagcagaaactttcttaccttaaaaaaaaaaaaaaaaaaaaaaaaaaaaaa

SEQ ID NO: 2 - DNA, 2547 bp, Homo sapiens
  misc_feature: SPLICE VARIANT
gttttgttaggcaaaaagagactattgaaagctgagactttagaattgagtgacttatatgttagtgataagaagaaggatatgtctccaccctttatttgtgaggagacagatgaacaaaagcttcaaactctagacatcggtagtaacttggagaaagaaaaattagagaattccagatccttagaatgcagatcagatccagaatctcctatcaaaaaaacaagtttatctcctacttctaaacttggatactcatatagtagagatctagaccttgctaagaaaaaacatgcttccctgaggcagacggagtctgatccagatgctgatagaaccactttaaatcatgcagatcattcatcaaaaatagtccagcatcgattgttatctagacaagaagaacttaaggaaagagcaagagttctgcttgagcaagcaagaagagatgcagccttaaaggcggggaataagcacaataccaacacagccaccccattctgcaacaggcagctaagtgatcagcaagatgaagagcgacgtcggcagctgagagagagagctcgtcagctaatagcagaagctcgatctggagtgaagatgtcagaacttcccagctatggtgaaatggctgcagaaaagttgaaagaaaggtcaaagcaaaacagtaagttggtggacttgaagctgaagaagctcctagaagttcagccacaggtggcaaattcaccctccagtgctgcccagaaagctgtaactgagagctcagagcaggacatgaaaagtggcacagaagatctccggactgaacgattacaaaaaacaacagaacgttttagaaatcctgttgtgttcagcaaagattctacagtcagaaaaactcaacttcagtctttcagccaatatattgagaatagaccagagatgaaaaggcagagatcaatacaggaagatacaaagaaaggaaatgaggagaaggcagcgataactgaaactcagaggaagccatcagaagatgaagtgcttaataaagggttcaaagacaccagtcagtatgtagtaggagaattggcagcactagagaatgagcaaaagcaaattgacacccgtgccgcgctggtggagaagcgccttcgctatctcatggacacaggaaggaacacagaagaagaagaagctatgatgcaggaatggtttatgttagttaataagaaaaatgccttaataaggagaatgaatcagctctctcttctggaaaaagaacatgatttagaacgacggtatgagctgctgaaccgggaattgagggcaatgctagccattgaagactggcagaagaccgaggcccagaagcgacgcgaacagcttctgctagatgagctggtggccctggtgaacaagcgcgatgcgctcgtcagggacctggacgcgcaggagaagcaggccgaagaagaagatgagcatttggagcgaactctggagcaaaacaaaggcaagatggccaagaaagaggagaaatgtgttcttcagtagccatcagatcagaaagaatctctcccaacattttagagtcttgcttcccaaaccagaaaaagtcagactcattgttgatttaaaacttttaacattttgtttggctggattgtactactttacctctactttaccaccaccacccttttcctccctcctttccaaataatatacagaactccaaaatagcttcatttaaggatttttttgtgagttaacaatttccttgaaatcctgtgaaatagatttgcacagacaccttgtgagtgattggtattggaggtgttcaagaaactgttcgaaaaagaacaaaaacacttccctcgttattttctctcattttttgatgagaggaaaatttgaaacattattcttgttgttgttggtaatagcataatgacagtgggaggggtacaaggggataagaaaaatgtcatgatttttttccggtcctgccacatgtaacacttactctgttacctaaattttatagttagatcatatccaatctacttattaaactgtgttctatttaccagtggagtttttctgcagtggttgcgtttcactgtaaggataatggagttcctctcctctgctttcctcagaggatggtcctttaacatagccagaaacaagccctgtggtttgaaggtgagctgtgaggatgggactaattgatatgcaccagtttacaaagacagtcttatcatccgagaatacaccatctttttctctggataattatttcttacatcatgcttgattcctacattttgttgggtttcaacattggctcacgaatgctgttaatatttattctgtattgataaaaagtctgtcttgccactacaagtaaatcccccatttaatattttcttctttagcatagcactgtcattttttgtgaaaatggttatgtttatttattacaatactgagtcatatataaattttcaataaaagcagaaactttcttaccttaaaaaaaaaaaaaaaaaaaaaaaaaaaaaa

SEQ ID NO: 3 - protein, 523 aa, Homo sapiens
  MISC_FEATURE: PHOSPHORYLATION SITES 9X PROTEIN KINASE, 15X CASEINE KINASE, 2X TYROSINE KINASE
  MISC_FEATURE: PRENYLATION SITE (CAAX-BOX)
MSPPFICEETDEQKLQTLDIGSNLEKEKLENSRSLECRSDPESPIKKTSLSPTSKLGYSYSRDLDLAKKKHASLRQTESDPDADRTTLNHADHSSKIVQHRLLSRQEELKERARVLLEQARRDAALKAGNKHNTNTATPFCNRQLSDQQDEERRRQLRERARQLIAEARSGVKMSELPSYGEMAAEKLKERSKASGDENDNIEIDTNEEIPEGFVVGGGDELTNLENDLDTPEQNSKLVDLKLKKLLEVQPQVANSPSSAAQKAVTESSEQDMKSGTEDLRTERLQKTTERFRNPVVFSKDSTVRKTQLQSFSQYIENRPEMKRQRSIQEDTKKGNEEKAAITETQRKPSEDEVLNKGFKDTSQYVVGELAALENEQKQIDTRAALVEKRLRYLMDTGRNTEEEEAMMQEWFMLVNKKNALIRRMNQLSLLEKEHDLERRYELLNRELRAMLAIEDWQKTEAQKRREQLLLDELVALVNKRDALVRDLDAQEKQAEEEDEHLERTLEQNKGKMAKKEEKCVLQ

SEQ ID NO: 4 - protein, 481 aa, Homo sapiens
  MISC_FEATURE: PHOSPHORYLATION SITES 9X PROTEIN KINASE, 15X CASEINE KINASE, 2X TYROSINE KINASE
  MISC_FEATURE: PRENYLATION SITE (CAAX-BOX)
MSPPFICEETDEQKLQTLDIGSNLEKEKLENSRSLECRSDPESPIKKTSLSPTSKLGYSYSRDLDLAKKKHASLRQTESDPDADRTTLNHADHSSKIVQHRLLSRQEELKERARVLLEQARRDAALKAGNKHNTNTATPFCNRQLSDQQDEERRRQLRERARQLIAEARSGVKMSELPSYGEMAAEKLKEEQNSKLVDLKLKKLLEVQPQVANSPSSAAQKAVTESSEQDMKSGTEDLRTERLQKTTERFRNPVVFSKDSTVRKTQLQSFSQYIENRPEMKRQRSIQEDTKKGNEEKAAITETQRKPSEDEVLNKGFKDTSQYVVGELAALENEQKQIDTRAALVEKRLRYLMDTGRNTEEEEAMMQEWFMLVNKKNALIRRMNQLSLLEKEHDLERRYELLNRELRAMLAIEDWQKTEAQKRREQLLLDELVALVNKRDALVRDLDAQEKQAEEEDEHLERTLEQNKGKMAKKEEKCVLQ

SEQ ID NO: 5 - DNA, 2559 bp, Homo sapiens
  misc_feature: SPLICE VARIANT
gttttgttaggcaaaaagagactattgaaagctgagactttagaattgagtgacttatatgttagtgataagaagaaggatatgtctccaccctttatttgtgaggagacagatgaacaaaagcttcaaactctagacatcggtagtaacttggagaaagaaaaattagagaattccagatccttagaatgcagatcagatccagaatctcctatcaaaaaaacaagtttatctcctacttctaaacttggatactcatatagtagagatctagaccttgctaagaaaaaacatgcttccctgaggcagacggagtctgatccagatgctgatagaaccactttaaatcatgcagatcattcatcaaaaatagtccagcatcgattgttatctagacaagaagaacttaaggaaagagcaagagttctgcttgagcaagcaagaagagatgcagccttaaaggcggggaataagcacaataccaacacagccaccccattctgcaacaggcagctaagtgatcagcaagatgaagagcgacgtcggcagctgagagagagagctcgtcagctaatagcagaagctcgatctggagtgaagatgtcagaacttcccagctatggtgaaatggctgcagaaaagttgaaagaaaggtcaaaggcatctggagaacaaaacagtaagttggtggacttgaagctgaagaagctcctagaagttcagccacaggtggcaaattcaccctccagtgctgcccagaaagctgtaactgagagctcagagcaggacatgaaaagtggcacagaagatctccggactgaacgattacaaaaaacaacagaacgttttagaaatcctgttgtgttcagcaaagattctacagtcagaaaaactcaacttcagtctttcagccaatatattgagaatagaccagagatgaaaaggcagagatcaatacaggaagatacaaagaaaggaaatgaggagaaggcagcgataactgaaactcagaggaagccatcagaagatgaagtgcttaataaagggttcaaagacaccagtcagtatgtagtaggagaattggcagcactagagaatgagcaaaagcaaattgacacccgtgccgcgctggtggagaagcgccttcgctatctcatggacacaggaaggaacacagaagaagaagaagctatgatgcaggaatggtttatgttagttaataagaaaaatgccttaataaggagaatgaatcagctctctcttctggaaaaagaacatgatttagaacgacggtatgagctgctgaaccgggaattgagggcaatgctagccattgaagactggcagaagaccgaggcccagaagcgacgcgaacagcttctgctagatgagctggtggccctggtgaacaagcgcgatgcgctcgtcagggacctggacgcgcaggagaagcaggccgaagaagaagatgagcatttggagcgaactctggagcaaaacaaaggcaagatggccaagaaagaggagaaatgtgttcttcagtagccatcagatcagaaagaatctctcccaacattttagagtcttgcttcccaaaccagaaaaagtcagactcattgttgatttaaaacttttaacattttgtttggctggattgtactactttacctctactttaccaccaccacccttttcctccctcctttccaaataatatacagaactccaaaatagcttcatttaaggatttttttgtgagttaacaatttccttgaaatcctgtgaaatagatttgcacagacaccttgtgagtgattggtattggaggtgttcaagaaactgttcgaaaaagaacaaaaacacttccctcgttattttctctcattttttgatgagaggaaaatttgaaacattattcttgttgttgttggtaatagcataatgacagtgggaggggtacaaggggataagaaaaatgtcatgatttttttccggtcctgccacatgtaacacttactctgttacctaaattttatagttagatcatatccaatctacttattaaactgtgttctatttaccagtggagtttttctgcagtggttgcgtttcactgtaaggataatggagttcctctcctctgctttcctcagaggatggtcctttaacatagccagaaacaagccctgtggtttgaaggtgagctgtgaggatgggactaattgatatgcaccagtttacaaagacagtcttatcatccgagaatacaccatctttttctctggataattatttcttacatcatgcttgattcctacattttgttgggtttcaacattggctcacgaatgctgttaatatttattctgtattgataaaaagtctgtcttgccactacaagtaaatcccccatttaatattttcttctttagcatagcactgtcattttttgtgaaaatggttatgtttatttattacaatactgagtcatatataaattttcaataaaagcagaaactttcttaccttaaaaaaaaaaaaaaaaaaaaaaaaaaaaaa

SEQ ID NO: 6 - protein, 487 aa, Homo sapiens [sequence omitted]

SEQ ID NO: 7 - DNA, 4911 bp, Homo sapiens
ggcgggggagccctccagaatacccatcatatagcccctgaggtggcatggtgatgtctccatgagggaaccccttcccacttcatactgtcacgtatatcatagtgttcttgactgggccattcatctaagatgggatttaccctgtgaaacagggagaagacttatggaccccaagcatcatttcaagttgaagttgagtttttaaaagccatccatgcaaagttcctttgctttggaccctctgcattattaaagctgctgtattgctaacccagaactgctccagtgtcttgactgatcatcatggcttcagtttggaagagactgcagcgtgtgggaaaacatgcatccaagttccagtttgtggcctcctaccaggagctcatggttgagtgtacgaagaaatggtaaccagataaactggtggtagtttggaccagaagaagccgaaggaagtcttctaaggcacatagctggcaacctggaataaaaaatccctatcgtggtgttgttgtgtggcctgttcctgaaaacattgaaatcactgtaacactttttaaggatcctcatgcggaagaatttgaagacaaagagtggacatttgtcatagaaaatgaatccccttctggtcgaaggaaagctcttgctactagcagcatcaatatgaaacagtatgcaagccctatgccaactcagactgatgtcaagttaaaattcaagccattatctaaaaaagttgtatctgccgctcttcagttttcattatcttgcatttttctgagggaaggaaaagccacagatgaagacatgcaaagtttggctagtttggtgagtatgaagcaggctgacattggcaatttagatgacttcgaagaagataatgaagatgatgatgagaacagagtgaaccaagaagaaaaggcagctaaaattacagagcttatcaacaaacttaactttttggatgaagcagaaaaggacttggccaccgtgaattcaaatccatttgatgatcctgatgctgcagaattaaatccatttggagatcctgactcagaagaacctatcactgaaacagcttcacctagaaaaacagaagactctttttataataacagctataatccctttaaagaggtgcagactccacagtatttgaacccattcgatgagccagaagcatttgtgaccataaaggattctcctccccagtctacaaaaagaaaaaatataagacctgtggatatgagcaagtacctctatgctgatagttctaaaactgaagaagaagaattggatgaatcaaatcctttttatgaacctaaatcaactcctcctccaaataatttggtaaatcctgttcaagaactagaaactgaaaggcgagtgaaaagaaaggccccggctccaccagtcctctcaccaaaaacaggagtattaaatgaaaacacagtttctgcaggaaaagatctctctacttctcctaagccaagccctataccaagtcctgttttggggcgaaagccaaatgctagtcagtctttgcttgtatggtgtaaagaagttacaaagaactaccgaggagtaaaaatcaccaattttactacatcgtggagaaatggtttatctttttgtgcaatattacaccactttagaccagatttaattgactacaagtctctgaatcctcaagatattaaagagaacaacaaaaaggcatacgatggatttgccagcataggaatttcccgattattggaaccttctgatatggtattattagcaattcctgataaactgactgttatgacttatctctatcaaataagggcacatttcagtggccaagaactaaatgtcgttcagatagaggaaaacagcagtaaaagcacatataaagttggaaactatgaaacagatacaaacagttctgttgatcaagaaaaattctatgcagagcttagtgatctgaagcgggagcctgaactacaacagcctatcagcggagcagtagacttcttatcacaggatgactctgtatttgtaaatgatagcggggttggagagtcagaaagtgagcatcaaactcctgatgatcaccttagtccaagcacagcctccccttactgtcgcaggactaaaagtgacacagaaccccagaagtctcagcagagctctggaaggacttcaggatctgatgaccctggaatatgttccaatacagattcaacccaagcacaggttttgttaggcaaaaagagactattgaaagctgagactttagaattgagtgacttatatgttagtgataagaagaaggatatgtctccaccctttatttgtgaggagacagatgaacaaaagcttcaaactctagacatcggtagtaacttggagaaagaaaaattagagaattccagatccttagaatgcagatcagatccagaatctcctatcaaaaaaacaagtttatctcctacttctaaacttggatactcatatagtagagatctagaccttgctaagaaaaaacatgcttccctgaggcagacggagtctgatccagatgctgatagaaccactttaaatcatgcagatcattcatcaaaaatagtccagcatcgattgttatctagacaagaagaacttaaggaaagagcaagagttctgcttgagcaagcaagaagagatgcagccttaaaggcggggaataagcacaataccaacacagccaccccattctgcaacaggcagctaagtgatcagcaagatgaagagcgacgtcggcagctgagagagagagctcgtcagctaatagcagaagctcgatctggagtgaagatgtcagaacttcccagctatggtgaaatggctgcagaaaagttgaaagaaaggtcaaaggcatctggagatgaaaatgataatattgagatagatactaacgaggagatccctgaaggctttgttgtaggaggtggagatgaacttactaacttagaaaatgaccttgatactcccgaacaaaacagtaagttggtggacttgaagctgaagaagctcctagaagttcagccacaggtggcaaattcaccctccagtgctgcccagaaagctgtaactgagagctcagagcaggacatgaaaagtggcacagaagatctccggactgaacgattacaaaaaacaacagaacgttttagaaatcctgttgtgttcagcaaagattctacagtcagaaaaactcaacttcagtctttcagccaatatattgagaatagaccagagatgaaaaggcagagatcaatacaggaagatacaaagaaaggaaatgaggagaaggcagcgataactgaaactcagaggaagccatcagaagatgaagtgcttaataaagggttcaaagacaccagtcagtatgtagtaggagaattggcagcactagagaatgagcaaaagcaaattgacacccgtgccgcgctggtggagaagcgccttcgctatctcatggacacaggaaggaacacagaagaagaagaagctatgatgcaggaatggtttatgttagttaataagaaaaatgccttaataaggagaatgaatcagctctctcttctggaaaaagaacatgatttagaacgacggtatgagctgctgaaccgggaattgagggcaatgctagccattgaagactggcagaagaccgaggcccagaagcgacgcgaacagcttctgctagatgagctggtggccctggtgaacaagcgcgatgcgctcgtcagggacctggacgcgcaggagaagcaggccgaagaagaagatgagcatttggagcgaactctggagcaaaacaaaggcaagatggccaagaaagaggagaaatgtgttcttcagtagccatcagatcagaaagaatctctcccaacattttagagtcttgcttcccaaaccagaaaaagtcagactcattgttgatttaaaacttttaacattttgtttggctggattgtactactttacctctactttaccaccaccacccttttcctccctcctttccaaataatatacagaactccaaaatagcttcatttaaggatttttttgtgagttaacaatttccttgaaatcctgtgaaatagatttgcacagacaccttgtgagtgattggtattggaggtgttcaagaaactgttcgaaaaagaacaaaaacacttccctcgttattttctctcattttttgatgagaggaaaatttgaaacattattcttgttgttgttggtaatagcataatgacagtgggaggggtacaaggggataagaaaaatgtcatgatttttttccggtcctgccacatgtaacacttactctgttacctaaattttatagttagatcatatccaatctacttattaaactgtgttctatttaccagtggagtttttctgcagtggttgcgtttcactgtaaggataatggagttcctctcctctgctttcctcagaggatggtcctttaacatagccagaaacaagccctgtggtttgaaggtgagctgtgaggatgggactaattgatatgcaccagtttacaaagacagtcttatcatccgagaatacaccatctttttctctggataattatttcttacatcatgcttgattcctacattttgttgggtttcaacattggctcacgaatgctgttaatatttattctgtattgataaaaagtctgtcttgccactacaagtaaatcccccatttaatattttcttctttagcatagcactgtcattttttgtgaaaatggttatgtttatttattacaatactgagtcatatataaattttcaataaaagcagaaactttcttaccttaaaaaaaaaaaaaaaaaaaaaaaaaaaaaa

SEQ ID NO: 8 - protein, 1076 aa, Homo sapiens
  MISC_FEATURE: PHOSPHORYLATION SITES 24X PROTEIN KINASE, 29X CASEINE KINASE, 5X TYROSINE KINASE
MKQYASPMPTQTDVKLKFKPLSKKVVSAALQFSLSCIFLREGKATDEDMQSLASLVSMKQADIGNLDDFEEDNEDDDENRVNQEEKAAKITELINKLNFLDEAEKDLATVNSNPFDDPDAAELNPFGDPDSEEPITETASPRKTEDSFYNNSYNPFKEVQTPQYLNPFDEPEAFVTIKDSPPQSTKRKNIRPVDMSKYLYADSSKTEEEELDESNPFYEPKSTPPPNNLVNPVQELETERRVKRKAPAPPVLSPKTGVLNENTVSAGKDLSTSPKPSPIPSPVLGRKPNASQSLLVWCKEVTKNYRGVKITNFTTSWRNGLSFCAILHHFRPDLIDYKSLNPQDIKENNKKAYDGFASIGISRLLEPSDMVLLAIPDKLTVMTYLYQIRAHFSGQELNVVQIEENSSKSTYKVGNYETDTNSSVDQEKFYAELSDLKREPELQQPISGAVDFLSQDDSVFVNDSGVGESESEHQTPDDHLSPSTASPYCRRTKSDTEPQKSQQSSGRTSGSDDPGICSNTDSTQAQVLLGKKRLLKAETLELSDLYVSDKKKDMSPPFICEETDEQKLQTLDIGSNLEKEKLENSRSLECRSDPESPIKKTSLSPTSKLGYSYSRDLDLAKKKHASLRQTESDPDADRTTLNHADHSSKIVQHRLLSRQEELKERARVLLEQARRDAALKAGNKHNTNTATPFCNRQLSDQQDEERRRQLRERARQLIAEARSGVKMSELPSYGEMAAEKLKERSKASGDENDNIEIDTNEEIPEGFVVGGGDELTNLENDLDTPEQNSKLVDLKLKKLLEVQPQVANSPSSAAQKAVTESSEQDMKSGTEDLRTERLQKTTERFRNPVVFSKDSTVRKTQLQSFSQYIENRPEMKRQRSIQEDTKKGNEEKAAITETQRKPSEDEVLNKGFKDTSQYVVGELAALENEQKQIDTRAALVEKRLRYLMDTGRNTEEEEAMMQEWFMLVNKKNALIRRMNQLSLLEKEHDLERRYELLNRELRAMLAIEDWQKTEAQKRREQLLLDELVALVNKRDALVRDLDAQEKQAEEEDEHLERTLEQNKGKMAKKEEKCVLQ

SEQ ID NO: 9 - DNA, 19 bp, Homo sapiens
ccatactcgacgacttggc

SEQ ID NO: 10 - DNA, 12 bp, Artificial
  Chemically Synthesized
ccgcgtggatcc

SEQ ID NO: 11 - DNA, 18 bp, Artificial
  chemically synthesized
ccgggaattcatcgtgac

SEQ ID NO: 12 - DNA, 24 bp, Artificial
  chemically synthesized
gactacaaggacgacgatgacaag

(19) United States
(12) Patent Application Publication
(10) Pub. No.: US 2007/0154911 A1
(43) Pub. Date: Jul. 5, 2007

(54) REGULATORY PROTEIN PKE#83 FROM HUMAN KERATINOCYTES

(76) Inventors: Michael Kramer, Pfungstadt (DE); Michael Bechtel, Schriesheim (DE); Jeanette Reinartz, Heidelberg (DE); Birgit Schaefer, Heidelberg (DE); Reinhard Wallich, Heidelberg (DE)

Correspondence Address: AKERMAN SENTERFITT, P.O. BOX 3188, WEST PALM BEACH, FL, 33402-3188, US

(21) Appl. No.: 11/566,115

(22) Filed: Dec. 1, 2006

(30) Foreign Application Priority Data
Nov. 26, 1998 (DE) 198 54 672.6
Dec. 7, 1998 (DE) 198 56 301.9
Nov. 19, 1999 (DE) PCT/DE99/03732

(51) Int. Cl. C12Q 1/68; C07H 21/04; C12P 21/06; C07K 14/78

(52) U.S. Cl. 435/6; 435/69.1; 435/320.1; 435/325; 530/350; 536/23.5

(57) ABSTRACT

The invention relates to an isolated polypeptide which is the same as or similar to (i.e., has the same function and effect as) a protein which occurs naturally in human keratinocytes and is more strongly expressed when the keratinocytes are in their activated state. The invention also relates to an isolated nucleic acid which codes a polypeptide or protein of this type that is typical for human keratinocytes and to the use of said polypeptide and said nucleic acid for detection, especially diagnostic purposes and/or for therapeutic purposes or the use of reagents, especially recombinant vector molecules and antibodies, against molecules of this type. The inventive protein has the amino acid sequence shown in sequence protocol SEQ ID NO:3 or an allele or derivative of this amino acid sequence produced therefrom by amino acid substitution, deletion, insertion, or inversion, and the inventive nucleic acid has either the amino acid sequence shown in sequence protocol SEQ ID NO:1 or a nucleotide sequence complementary thereto or a partial sequence of one of these two nucleotide sequences or a nucleotide sequence which is completely or partially hybridizable one of these two nucleotide sequences.

This application claims priority to and incorporates by reference German Patent Application No. 198 54 672.6 filed Nov. 26, 1998 and German Patent Application No. 198 56 301.9 filed Dec. 7, 1998 through International Application No. PCT/DE99/03732 filed Nov. 19, 1999, and U.S. application Ser. No. 09/856,723 filed Sep. 17, 2001, of which this application is a division.

### BACKGROUND INFORMATION

The invention relates to an isolated polypeptide identical or similar (i.e., the same in function and effect) to a protein that occurs naturally in keratinocytes and is increasingly expressed when the keratinocytes are in an activated state. It also relates to an isolated nucleic acid, which encodes a polypeptide or protein typical for human keratinocytes, and to the use of this polypeptide and this nucleic acid for detection, in particular diagnostic, and/or therapeutic purposes, and reagents manufactured with the use of at least one of these molecules, in particular recombinant vector molecules and antibodies.

Based on prior art as currently exists, essentially pharmaceuticals with a broad range of action are used in skin treatment to influence epidermal disturbances, e.g., autoimmune dermatoses “Pemphigus vulgaris” and “Bullous Pemphigoid”, in particular locally or systemically applied glucocorticoids, vitamin A acid derivatives, antimetabolites and cytostatics, or more or less non-specific measures are used in treatment, such as “dye therapy” or “light therapy”. However, the disadvantage to all known agents or measures is that they are not very specific, and hence of course bring about numerous side effects.

The preparation of more specific agents has thus far been unsuccessful due to a basic problem that has persisted in dermatology for a long time, namely that the number of cellular target molecules, hereinafter generally referred to as target structures (“targets”), which might serve as a point of attack for exerting a (specific) influence on cellular metabolism, in particular from a medical or even cosmetic standpoint, is narrowly restricted in epidermal keratinocytes.

Therefore, the object of this invention is to provide new target structures in epidermal keratinocytes that can serve as a point of attack for diagnostic, therapeutic and cosmetic agents, or generally for influencing cellular metabolism.

### SUMMARY OF THE INVENTION

One solution to this object involves preparing a polypeptide or protein of the kind mentioned at the outset, which is upwardly adjusted in activated keratinocytes, i.e., increasingly expressed or produced, and kept at a higher concentration level, and which has the amino acid sequence indicated in either the SEQ ID NO:3 or SEQ ID NO:4 sequence protocol or the SEQ ID NO:6 or SEQ ID NO:8 sequence protocol, or an allele or derivative of this amino acid sequence obtained through amino acid substitution, deletion, insertion or inversion. In the following, the polypeptides with the SEQ ID NO:3 or SEQ ID NO:4 or the SEQ ID NO:6 or SEQ ID NO:8 amino acid sequence shall also be referred to as protein pKe#83.

Another solution to this object involves preparing an isolated nucleic acid that codes a protein, which is identical or similar to a protein that occurs naturally in human keratinocytes and is increasingly expressed when the keratinocytes are in an activated state, and which has the nucleotide sequence indicated in either the SEQ ID NO:1 sequence protocol or the SEQ ID NO:7 sequence protocol, or a nucleotide sequence complementary thereto, or a partial sequence of one of these two nucleotide sequences, or a nucleotide sequence that hybridizes wholly or in part with one of these two nucleotide sequences, wherein “U” can take the place of “T” in these sequence protocols SEQ ID NO:1 and SEQ ID NO:7. This group of nucleic acids or nucleotide sequences according to the invention also includes in particular splice variants (e.g., SEQ ID NO:2 or SEQ ID NO:5) and sense or antisense oligonucleotides, which hybridize with the nucleotide sequence indicated in the SEQ ID NO:1 sequence protocol or the SEQ ID NO:7 sequence protocol, preferably identical or (partially) complementary to the latter. Two preferred splice variants of the inventive nucleotide sequence according to SEQ ID NO:1 and SEQ ID NO:7 are indicated in the SEQ ID NO:2 and SEQ ID NO:5 sequence protocols.

As a result, the invention also encompasses proteins or polypeptides of the kind mentioned at the outset, which have an amino acid sequence that results from such a splice variant, in particular the splice variant of an mRNA, which is identical or wholly or partially complementary to the nucleotide sequence indicated in the SEQ ID NO:1 sequence protocol or the SEQ ID NO:7 sequence protocol.

The sense or antisense oligonucleotides according to the invention encompass at least 6, preferably 8 to 25 nucleotides.

The term “hybridized” relates to the hybridization procedures known in the art under conventional, in particular also under highly stringent hybridization conditions. The expert selects the specific hybridization parameters based on the used nucleotide sequence and his or her general technical knowledge (compare: Current Protocols in Molecular Biology, Vol. 1, 1997, John Wiley & Sons Inc., Suppl. 37, Chapter 4.9.14).

In addition to the nucleotide sequences indicated in sequence protocols SEQ ID NO: 1, SEQ ID NO: 7, SEQ ID NO: 2 and SEQ ID NO: 5 and the nucleotide sequences corresponding to these sequences in terms of genetic code degeneration, this invention also encompasses those nucleotide sequences that hybridize with them under stringent conditions. In this invention, the term “hybridize” or “hybridization” is used as in Sambrook et al., Molecular Cloning, A Laboratory Manual, Cold Spring Harbor, Laboratory Press, 1989, 1.101 to 1.104. According to this publication, hybridization under stringent conditions exists when a positive hybridization signal is still observed after washing for one hour with 1×SSC and 0.1% SDS, preferably with low-concentrated SSC, in particular 0.2×SSC, at a temperature of at least 55° C., preferably 62° C. and especially preferred 68° C. Each nucleotide sequence that hybridizes under such washing conditions with a nucleotide sequence according to SEQ ID NO: 1, SEQ ID NO: 7, SEQ ID NO: 2 or SEQ ID NO: 5 or with one having the sequence according to SEQ ID NO: 1 or SEQ ID NO: 7 or SEQ ID NO: 2 or SEQ ID NO: 5 within the framework of degeneration of the nucleotide sequence corresponding to the genetic code belongs to the subject matter of the present invention.

The nucleic acid(s) according to the invention can be obtained from both a natural source or synthetically or semi-synthetically. Its presentation as cDNA has proven to be particularly effective in practice.

The polypeptide that has the amino acid sequence according to SEQ ID NO:3 or SEQ ID NO:8 and is coded by the nucleic acid indicated in the SEQ ID NO:1 or SEQ ID NO:7 sequence protocol, and that is referred to as protein pKe#83 below, is upwardly adjusted in human epidermal keratinocytes, namely increasingly expressed (produced) and kept at a significantly higher concentration level in comparison to the initial state if these cells are in the “activated” state, i.e., in a state of proliferation and/or migration, among others, e.g., after an accidental skin injury or given the autoimmunologically induced bullous dermatoses “Pemphigus vulgaris” (triggered by autoantibodies against desmosomes) and “Bullous Pemphigoid” (triggered by autoantibodies against hemidesmosomes). The activated state of the human epidermal keratinocytes is also manifested in an elevated expression of known activation markers uPA (urokinase-type plasminogen activator) and uPA-R (receptor for urokinase-type plasminogen activator) relative to the resting state (initial state), and can be qualitatively and quantitatively detected based on these markers. (compare: Schäfer, et al., 1996: Dispase-mediated basal detachment of cultured keratinocytes induces urokinase-type plasminogen activator (uPA) and its receptor (uPA-R, CD87), Exp. Cell Res. 228, pp. 246-253).

Protein pKe#83 has a so-called prenyl-group binding site (“CAAX Box”). This is a binding site that allows a post-translational change of numerous eukaryotic proteins by appending a farnesyl or gerany-geranyl group to a cysteine residue that is three amino acids away from the C terminal, wherein the two amino acids situated at the C terminal are generally aliphatic. Ras proteins and numerous G proteins have such a CAAX box.

In addition, the “pKe#83” protein has several putative phosphorylation sites. The cited motifs indicate that the pKe#83 protein is involved in signal transduction processes.

The (isolated) preparation of protein pKe#83, namely the description of nucleotide sequences that code this protein, and the indication of (one of) its amino acid sequence(s) make it possible to exert a targeted influence on the metabolism of physiologically active or activated keratinocytes, and of course of other cells that express protein pKe#83, in particular for purposes of medical therapy and cosmetic treatment.

The invention also relates to recombinant DNS vector molecules, which encompass a nucleic acid according to the invention, and which have the ability to express a protein that occurs in human keratinocytes and is increasingly expressed when the keratinocytes are in an activated state, in particular protein pKe#83, in a prokaryotic or eukaryotic cell. These DNS vector molecules preferably involve derivatives of the plasmid pUEX-1 and/or the plasmid pGEX-2T and/or the plasmid pcDNA3.1, since these vectors have proven to be highly suitable in practice. Especially preferred are the vector construct pGEX-2T-pKe#83 according to the vector protocol disclosed on FIG. 2, and the vector construct pcDNA3.1/pKe#83-FLAG according to the vector protocol disclosed on FIG. 3. While the eukaryotic cell includes in particular cells from cell cultures, e.g., COS cells, the respective cell can just as well also be a constituent of a living organism, e.g., a transgenic mouse.

Therefore, the invention also encompasses transformed host cells that contain a nucleic acid according to the invention that is linked with an activatable promotor, which is contained in these cells naturally or as the result of recombination, and that (consequently) have the ability to express a protein that occurs naturally in human keratinocytes and is increasingly expressed when the keratinocytes are in an activated state, in particular protein “pKe#83”.

The invention also relates to the use of a nucleic acid according to the invention or a vector molecule according to the invention to manufacture transgenic mammals, in particular mice or rats.

The transfectants according to the invention open up an opportunity for research and development work aimed at further clarifying the protein “pKe#83”-induced changes in cell morphology and cellular base functions such as proliferation, adhesion, migration and differentiation, in particular with an eye toward answering the question as to whether protein “pKe#83” itself possesses a “pathogenic” activity.

The object of this invention also relates to a reagent for the indirect detection of a protein that is encountered in human keratinocytes and increasingly expressed when the keratinocytes are in an activated state, in particular protein “pKe#83”, wherein this reagent is characterized by the fact that it encompasses at least one nucleic acid according to the invention. In this context, “for the indirect detection” implies that the protein-coding mRNA is actually directly detected, and hence the protein is only indirectly detected (by means of this mRNA).

Protein “pKe#83” and the polypeptides related thereto, i.e., to the amino acid sequence indicated in the SEQ ID NO:3 sequence protocol or SEQ ID NO:8 sequence protocol, i.e., the polypeptides that can be derived through substitution, deletion, insertion and/or inversion from the amino acid sequence according to SEQ ID NO:3 or SEQ ID NO:8, or that have an amino acid sequence resulting from a splice variant of an mRNA, which is identical or complementary to the nucleotide sequence according to the SEQ ID NO:1 sequence protocol or the SEQ ID NO:7 sequence protocol, or to a partial sequence of thereof, or at least hybridized, offer numerous applications in the area of dermatological research and development. In particular, antibodies can be developed against these polypeptides or proteins, which then can be correspondingly modified for use either as diagnostic or therapeutic agents, or as cosmetic agents (“cosmeceuticals”).

Consequently, the invention also encompasses the use of such a protein or polypeptide for manufacturing a (monoclonal or polyclonal) antibody against this polypeptide, the aforementioned antibody itself, and also its use for the diagnostic and/or therapeutic treatment of dermatological diseases, for the cosmetic treatment of the epidermis, and for the diagnostic and/or cosmetic treatment of other tissues or organs that express protein “pKe#83”.

According to more recent scientific knowledge, sense and/or antisense oligonucleotides are also possible as active agents for pharmacotherapy (compare G. Hartmann et al. 1998: Antisense Oligonucleotides, Deutsches Ärzteblatt 95, Issue 24, C1115-C1119), and also as active agents with a fundamentally new operating principle in pharmacotherapy.

Therefore, the present invention also relates to the use of sense or antisense oligonucleotides according to the invention for diagnostic and/or therapeutic treatment, in particular of dermatological diseases, or for the cosmetic treatment in particular of the epidermis.

One technically and economically important potential application for a polypeptide according to the invention or a nucleic acid according to the invention also involves not least the fact that such a molecule can be used in a screening procedure to isolate materials from a very high number of provided materials that specifically bind to the respective nucleic acid or respective polypeptide. These substances can then serve as the parent material (lead structure) for the development of substances for use in pharmacology, and hence offer the preconditions for the development of alternative pharmaceuticals for diagnosis and therapy, in particular with respect to the dermatological diseases mentioned at the outset and/or other diseases in which protein “pKe#83” plays an important role.

In this regard, the invention also relates to the application of a polypeptide according to the invention or a nucleic acid according to the invention for identifying substances that can be used in pharmacology, which bind to the polypeptide or nucleic acid, thereby influencing its/their function and/or expression, in particular exerting an inhibiting or activating effect.

### BRIEF DESCRIPTION OF THE DRAWINGS

The invention will be explained in greater detail below based on manufacturing and application examples. The figures mentioned in conjunction with these examples show:

FIG. 1: an rt-PCR-detection of “pKe#83 ”-specific mRNA

FIG. 2: the vector construct pGEX-2T/pKe#83 (the nucleotide sequences are shown in SEQ ID NOs: 10 and 11 respectively, in order of appearance).

FIG. 3: the vector construct pcDNA3.1/pKe#83-FLAG (the nucleotide sequence is shown in SEQ ID NO: 12).

FIG. 4: an immunoblot detection of recombinant pKe#83 protein in E. Coli cells after transfection with the vector construct pGEX-2T/pKe#83

FIG. 5: an immunoblot detection of recombinant pKe#83 protein in Cos cells after transfection with the vector construct pcDNA3.1/pKe#83-FLAG

FIG. 6: an immunoblot detection of anti-protein pKe#83 antibodies from rabbit serum on recombinant pKe#83 protein (A) and an immunoblot detection of expressed protein pKe#83 in transfected Cos cells with antiprotein pKe#83 antibodies from rabbit serum (B).

FIG. 7: a “sandwich”-ELISA test using antibodies directed against the pKe#83 protein.

FIG. 8: an immune fluorescence test using rabbit “anti-pKe#83 IgG” on normal skin sections (C), NHEK sheets directly after dispase-induced detachment and (A) and NHEK sheets 8 hours after dispase-induced detachment (B).

FIG. 9: Keratinocytes (HaCaT cells) after treatment with pKe#83-specific antisense oligonucleotides (B) and control oligonucleotides (A)

### DETAILED DESCRIPTION

### EXAMPLE 1

### Manufacture of Protein pKe#83

A) Extraction or Manufacture of a Polynucleotide that Codes Protein “pKe#83”

The polynucleotide source consisted of human epidermal keratinocytes of a cell culture or cell culture model described extensively in the publication of Schäfer B. M. et al., 1996: Dispase mediated basal detachment of cultured keratinocytes induces urokinase-type plasminogen activator (uPA) and its receptor (uPA-R, CD87), Exp. Cell Res. 228, pp. 246-253. Reference is hereby made expressly to the content of this publication. This cell culture or cell culture model is characterized by the fact that it makes it possible to convert keratinocytes from the resting [uPA⁻/uPA-R⁻] to the activated [uPA⁺/uPA-R⁺] state through enzymatic disruption of the cell/matrix contacts, i.e., dispase-induced detachment of the keratinocytes from the culture matrix. The induction of the activated state is reversible: the (renewed) formation of a confluent (=grown to maximal density), multilayered, keratinocyte “sheet” consisting of differentiated keratinocytes results in the downward adjustment of uPA and uPA-R, i.e., the slowing of production and setting to a lower concentration level (see the publication of Schäfer B. M, et al., 1996: Differential expression of urokinase-type plasminogen activator (uPA), its receptor (uPA-R), and inhibitor type-2 (PAI-2) during differentiation of keratinocytes in an organotypic coculture system, Exp. Cell Res. 220, pp. 415-423).

Cells in this cell culture or cell culture model shall also be referred to as NHEK below (=“normal human epidermal keratinocytes”).

The following measures were implemented for preparing the cell culture or cell culture model: NHEK obtained from a skin biopsy were trypsinated overnight at 4° C. and then cultivated in Petri dishes or 175 cm² culture flasks according to the “feeder-layer” technique of J. G. Rheinwald und H. Green (1975, Serial cultivation of strains of human epidermal keratinocytes: the formation of keratinizing colonies from single cells, Cell 6, pp. 331-334) for a duration of 8 days in Dulbecco's modified Eagle's Medium (DMEM) with a content of 10% (vol./vol.) fetal calf serum (FCS) and added adenine hemisulfate, insulin, transferrin, triiodothyronine, hydrocortisone, Forskolin, epidermal growth factor (EGF) and antibiotics (penicillin, streptomycin and gentamycin) under differentiation conditions, namely elevated calcium levels (37° C., 7% CO₂). Therefore, cultivation took place under conventional conditions common in prior art. Under these conditions, keratinocytes form confluent two to three-layer “epidermis equivalents”, or keratinocyte “sheets”.

These epidermis equivalents or keratinocyte sheets were detached from the culture matrix in a 30-minute treatment with dispase II (2.4 mg/ml in DMEM without FCS), washed twice in DMEM and then incubated in complete, conditioned DMEM for a duration of 4 or 8 hours. Incubation in conditioned DMEM took place to preclude the influence of fresh FCS. During incubation, the expression of known activation markers uPA and uPA-R was upwardly adjusted in these floating keratinocyte sheets, as was protein pKe#83 described for the first time herein. The uPA/uPA-R upward adjustment could be detected by means of known techniques, such as enzyme linked immunosorbent assay (ELISA), in-situ hybridization and immune fluorescence. The total RNA was extracted from the incubated cells (“RNA-Clean” kit, AGS company in Heidelberg) using the guanidinium-thiocyanate-phenol-chloroform extraction method known in the art (compare Chromczynski P. and Sacchi N., 1986: Single-step method of RNA isolation by acid guanidinium thiocyanate-phenol-chloroform extraction. Anal. Biochem. 162: pp. 156-159). The mRNA was isolated from the total RNA through binding on poly-T coated microbeads. This mRNA was used as the starting material for the ensuing step of subtraction cloning.

mRNA was isolated from adherent keratinocyte sheets for use in control tests or for comparison preparations, specifically according to the same procedural pattern described above, except that a dispase inhibitor, e.g., phosporamidone (100 μg/ml), was additionally applied to the dispase for the duration of dispase treatment.

The principle of subtraction cloning was used to establish a gene bank, which preferably contained cDNA of the dyshesion-induced gene, i.e., of those genes that were increasingly expressed after detachment of the keratinocyte sheets in the latter (or their cells). To this end, the mRNA obtained from the cells of the adherent keratinocyte sheets was again bound to poly-T coated microbeads, rewritten into single-strand cDNA on the latter, and then hybridized against the mRNA of detached, i.e., non-adherent keratinocyte sheets. Those mRNA molecules that were expressed only in the non-adherent state, i.e., after dyshesion, and hence found no hybridization partner, remained behind as a supernatant. They were rewritten into cDNA and cloned into the cloning vector pUEX-1.

For purposes of verification, the resultant gene bank was then also subjected to a southernblot procedure with [³²P]-marked cDNA of adherent and non-adherent keratinocyte sheets. Those cDNAs, or rather the host cell clones containing them, here the E. coli strain MC1061, which exhibited a distinct upward adjustment after dyshesion, were subsequently cultivated or multiplied overnight at 30° C. under conventional culture conditions. The plasmid DNA (pUEX1-cDNA) were prepared from these E. coli clones, and the cDNA fragments cut out of the pUEX1 vector were [³²P]-marked by means of random priming. The marked cDNA was used as a probe in northernblots with RNA from adherent and non-adherent keratinocyte sheets. The clones containing cDNA, which revealed no or only a slight signal with the RNA of adherent keratinocytes when used as a probe in the northernblot procedure, but exhibited a distinct signal with RNA of non-adherent keratinocytes, were selected for the ensuing step of sequencing.

Upon sequencing the respective clones by means of “non-radioactive cycle sequencing”, which is a modification of the sequencing method according to Sanger (F. Sanger et al., 1977: DNA sequencing with chain terminating inhibitors, Proc Natl Acad Sci USA 74: 5463-5467) and has in the meantime become a common method in prior art, the gene with the nucleotide sequence according to sequence protocol SEQ ID NO:1 and according to sequence protocol DEQ ID NO:7 was found, among others. In addition, the splice variants indicated in protocols SEQ ID NO:2 and SEQ ID NO:5 were found. The gene with the nucleotide sequence according to protocol SEQ ID NO:1 or SEQ ID NO:7 and the accompanying protein were designated pKe#83.

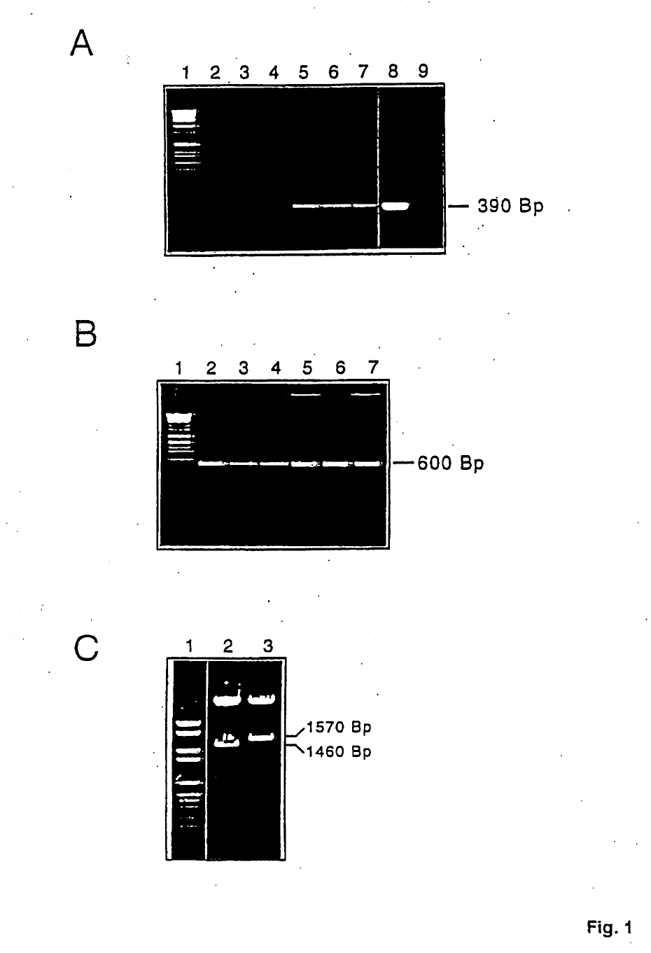

More detailed analyses of the mRNA that belongs to gene pKe#83, i.e., is pKe#83-specific mRNA (from dissolved, i.e., non-adherent keratinocyte sheets), provided information as to the fact that this mRNA has a size of about 2.6 kb (SEQ ID NO:1) to about 4.9 kb (SEQ ID NO:7). The nucleotide sequence according to SEQ ID NO:1 and SEQ ID NO:7 has a stop codon at the 3′ end at position 1651-1653 (SEQ ID NO:1) or at position 3895-3897 (SEQ ID NO:7) respectively, which stipulates the probable location of the transcription end. A so-called polyadenylation site is located at position 2612-2617 according to SEQ ID NO:1 or position 4856-4261 according to SEQ ID NO:7, respectively, exactly 26 nucleic acids before the poly-A site. A splice variant (SEQ ID NO:2) 111 nucleic acids (position 669-780 according to SEQ ID NO:1) shorter was discovered, as well as a second splice variant (SEQ ID NO:5) 108 nucleic acids (position 670-777 according to SEQ ID NO:1) shorter. FIG. 1C shows the result of cloning the pKe#83 overall cDNA sequence, wherein:
Trace 1=DNA molecular weight marker VI (154-2176 Bp, Boehringer Mannheim),
Trace 2=SEQ ID NO: 1 (1570 Bp)
Trace 3=SEQ ID NO: 2 (1460 Bp).

The polymerase chain reaction was used to show that the pKe#83-specific mRNA is upwardly adjusted following the dispase-induced detachment of the NHEK. FIG. 1A shows the result of a polymerase chain reaction after reverse transcription (rt-PCR) of the mRNA into cDNA and amplification with pKe#83-specific oligonucleotide primers. This result indicates that only a low amount of pKe#83-mRNA is present, or at least detectable, immediately after detachment of the NHEK, but a distinct upward adjustment could already be discerned 2 hours later.

B) Derivation of the Amino Acid Sequence and Characterization of the pKe#83 Protein by Means of the Polynucleotide Coding Therefore

Based on the genetic code of the “pKe#83”-cDNA and using a computer-assisted procedure (program: “HUSAR” [=Heidelberg Unix Sequence Analysis Resources], Version 4.0, German Cancer Research Center, Heidelberg, 1997), an amino acid sequence indicated in the SEQ ID NO:3 and SEQ ID NO:8 sequence protocols was derived from the nucleotide sequence according to sequence protocol SEQ ID NO:1 and sequence protocol SEQ ID NO:7. A structural analysis of these amino acid sequences according to the SEQ ID NO:3 and SEQ ID NO:8 sequence protocol with this very program yielded the following information.

From the amino acid composition of the pKe#83 protein a molecular weight of 60380 Da (according to SEQ ID NO:3 ) and 122180 Da (according to SEQ ID NO:8), respectively, with an isoelectric point of pH 5.3 (according to SEQ ID NO:3 ) and pH 4,9 (according to SEQ ID NO:8), respectively, is calculated.

The pKe#83 protein has a so-called prenyl-group binding site (“CAAX box”) and a series of possible phosphorylation sites (9× protein kinase C, 15× casein kinase II, 2× tyrosine kinase according to SEQ ID NO:3 and 24× protein kinase C, 29× casein kinase II, 5× tyrosine kinase according to SEQ ID NO:8). The cited motifs indicate that the pKe#83 protein is involved in signal transduction processes. Furthermore the protein pKe#83 according to SEQ ID NO:8 has some (eight) myristylation sites.

### EXAMPLE 2

### Detection of “pKe#83 ”-Specific mRNA in Cells Via Reverse Polymerase Chain Reaction

The polymerase chain reaction after reverse transcription (rt-PCR) was used to detect pKe#83-specific mRNA in cells (NHEK) of keratinocyte sheets after dispase treatment and in HaCaT cells. To this end, RNA was isolated from cells of keratinocyte sheets after dispase treatment and incubation for various intervals of time, and from HaCaT cells using standard methods (guanidinium-thiocyanate-phenol-chloroform extraction method) and rewritten to cDNA according to standard methods. This cDNA was subjected to a PCR, during which a partial fragment of 388 kb was amplified from the pKe#83-specific cDNA. A combination of the primers “pKe#83-forward 10” (¹⁰³²GAATAGACCAGAGATGAAAAGGCAG¹⁰⁵⁶)(residues 1032-1056 of SEQ ID NO: 1) and “pKe#83-reverse 17” (¹⁴¹⁸CGGTTCAGCAGCTCATACC¹³⁹⁹)(SEQ ID NO: 9) was used as the primer pair. 10 ng of cDNA were mixed with 10 μmM of primer along with a mixture of heat-stable DNA polymerase, ATP, TTP, GTP, CTP and polymerase buffer (e.g., compare: Current protocols in Molecular Biology, Vol. 1, 1997, John Wiley & Sons. Inc, Suppl. 37, Chapter 15), in this example in the form of the commercially available, ready-to-use “PCR master mix” from Clontech. In addition, the following control tests were performed: 1. The batch described above with the plasmid pUEX-1/pKe#83 instead of the cDNA (“positive control”); 2. The reaction batch described above without added cDNA (“negative control”); 3. The batch described above with GAPDH-specific primers (#302047, stratagenes; “GAPDH control”).

The reaction products of the PCR reaction were electrophoretically fractionated in agarose gel. FIG. 1A shows the result of a pKe#83-specific PCR fractionation. The following applies:
Trace 1=DNA molecular weight marker VII (359-8576 Bb, Boehringer Mannheim)
Trace 2=HaCaT
Trace 3=HMEC (cell line in which pKe#83 is not detectable)
Trace 4=NHEK T0 (immediately after detachment),
Trace 5=NHEK T2 (2 h after detachment)
Trace 6=NHEK T4 (4 h after detachment)
Trace 7=NHEK T8 (8 h after detachment),
Trace 8=pUEX/pKe#83-plasmid
Trace 9=no cDNA.

A PCR product of the expected size of ≈390 Bp was detected in traces 2,5-8, meaning that pKe#83-specific mRNA was detected in the keratinocyte sheets (NHEK) at times 2, 4 and 8 hours after dispase-induced detachment, and also in HaCaT cells.

FIG. 1.B shows the result of a GAPDH-specific PCR. The following applies:
Trace 1=DNA molecular weight marker VII (359-8576 Bb, Boehringer Mannheim)
Trace 2=HaCaT
Trace 3=HMEC
Trace 4=NHEK T0
Trace 5=NHEK T2
Trace 6=NHEK T4
Trace 7=NHEK T8

This GAPDH-specific PCR (“GAPDH-control”) proves that a negative PCR result in the pKe#83-specific batch cannot be attributed to the lack of cDNA, since a PCR product of the expected size of 600 Bp was detectable in all reaction times of T0-T8.

The rt-PCR makes it possible to detect pKe#83 expression even in cases where the pKe#83 protein cannot be detected using immunohistological methods, ELISA or immunoblot procedures due to an excessively low expression level.

### EXAMPLE 3

### Manufacture of Vector Molecules With the Ability to Express the Protein pKe#83 in Prokaryotic or Eukaryotic Cells, and Production and Purification of the Recombinant pKe#83 Protein

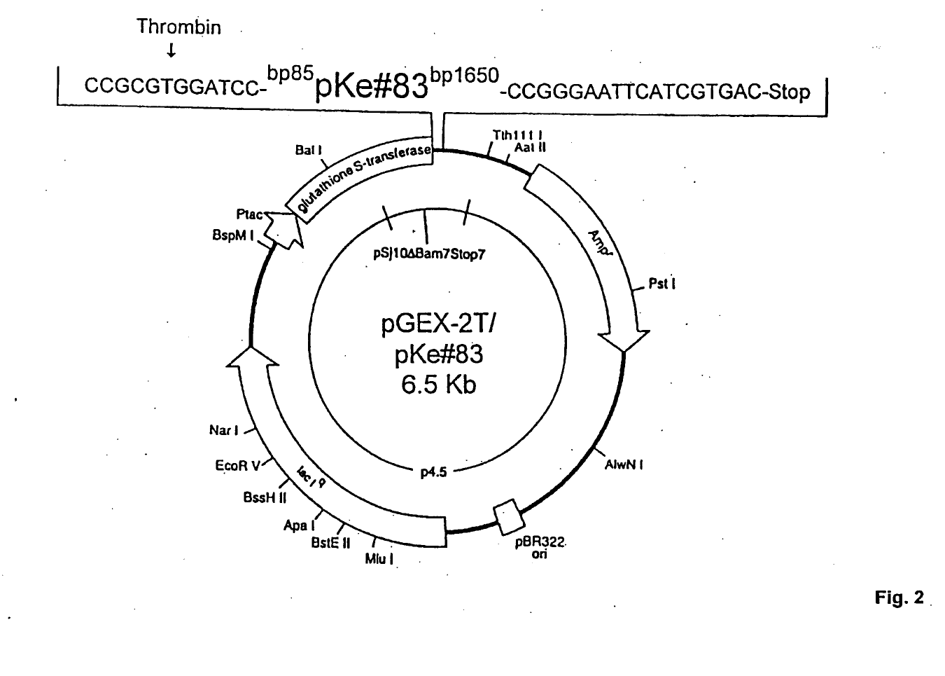

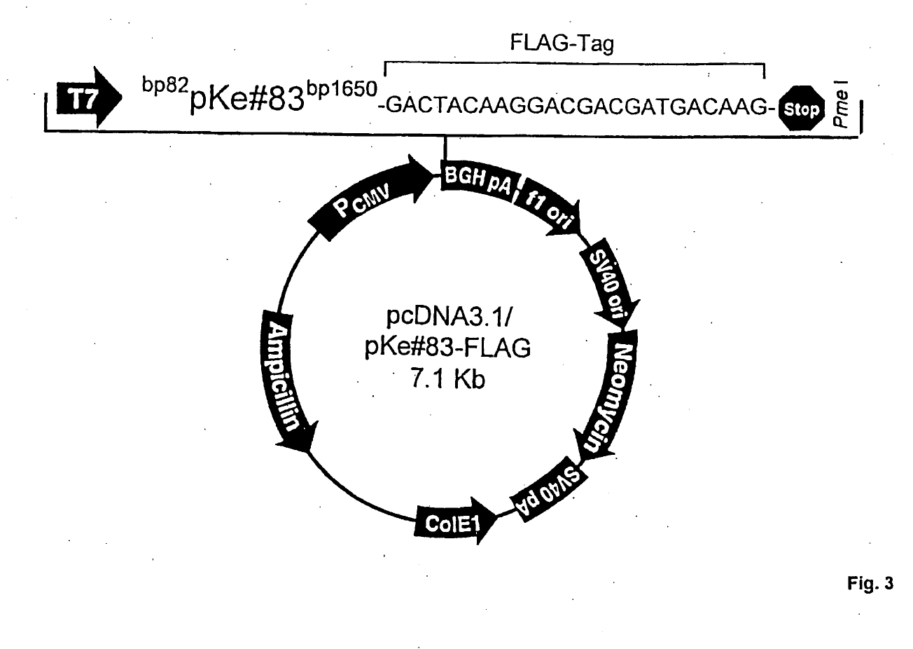

Two approaches were taken to manufacture or express the recombinant pKe#83 protein. In the first, the vector construct pGEX-2T/pKe#83 was fabricated according to vector protocol on FIG. 2 for expression in bacteria (E. coli DH5α). In the second, the vector construct pcDNA3.1/pKe#83-FLAG according to vector protocol on FIG. 3 was manufactured for purposes of expression in eukaryotic cells (Cos cells).

The vector construct pGEX-2T/pKe#83 was used according to standard protocols of the transformation of E.coli DH5α. The pKe#83 glutathion-S transferase (GST) fusion protein was expressed in bacteria, and the bacterial lysate was analyzed in an immunoblot procedure with anti-GST antibodies, specifically in comparison to the lysate of bacteria that were transformed with a control plasmid (no GST).

The pKe#83/GST fusion protein was washed out of the bacterial lysates through affinity chromatography using glutathion-sepharose 4B. The fractions from this purification were then analyzed with anti-GST antibodies in the immunoblot procedure.

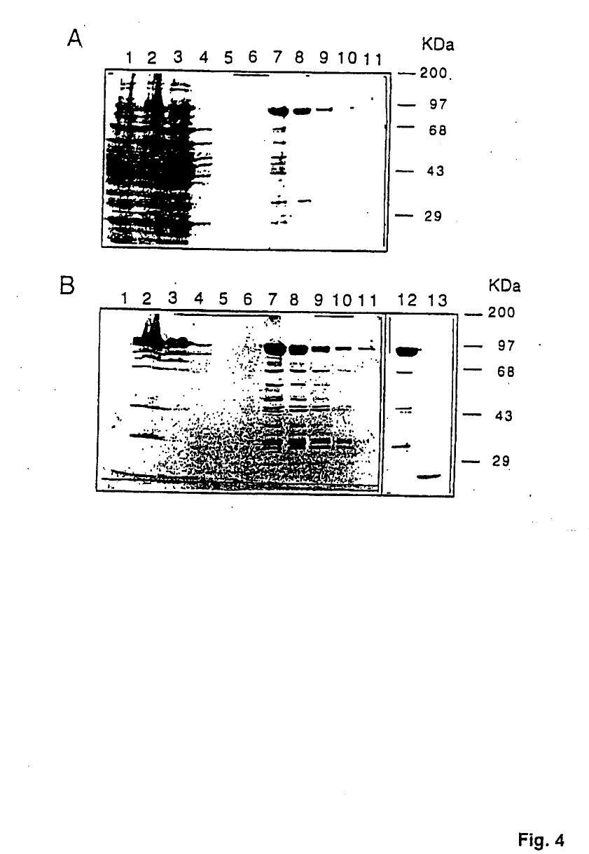

FIG. 4.B shows the product obtained form the immunoblot procedure, while FIG. 4.A depicts the corresponding protein stain (Ponceau red) of the blot before antibody staining. The following applies:
Trace 1=Bacterial lysate of the control transfectants
Trace 2=Bacterial lysate of the pUEX-2T/pKe#83-GST transfectants
Trace 3=Passage through column
Trace 4-6=Washing fraction 1-3
Trace 7-11=Elution fraction
Trace 12=pKe#83/GST fusion protein before thrombin digestion
Trace 13=pKe#83/GST fusion protein after thrombin digestion

The pKe#83/GST fusion protein had an apparent molecular weight of approx. 90 KDa. This allows us to conclude that the 90 KDa pKe#83/GST fusion protein consists of the GST protein (approx. 26 kDa) and an approx. 60-65 KDa large fragment of the pKe#83 protein.

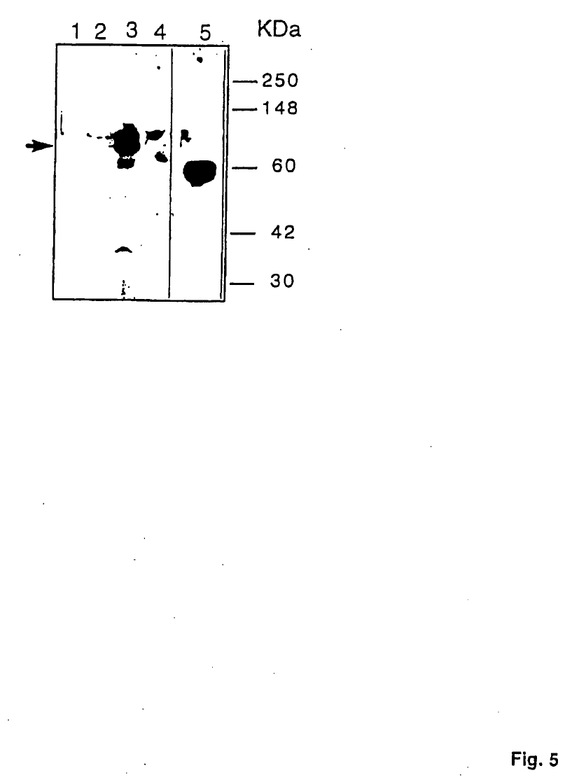

In the eukaryotic system, the pcDNA3.1/pKe#83-FLAG vector (FIG. 3) was transformed into so-called cos cells, i.e., into cells of the cos-cell line generally known in prior art. The cells were made to absorb the plasmid-DNA in a standard procedure through treatment with DEAE-dextran/chloroquine. The transformed cells were then incubated for three days under standard conditions (37° C. and 7% CO₂). The cos cells were subjected to lysis and analyzed in the immunoblot procedure using an antibody against the FLAG epitope. FIG. 5 shows the product of the immunoblot:
Trace 1=Cos cells transfected with pcDNA3.1/pKe#83-FLAG vector construct, developed with an isotope-identical control antibody,
Trace 2=Cos cells transfected with the pcDNA3.1 vector (without pKe#83), developed with an isotope-identical control antibody,
Trace 3=Cos cells transfected with pcDNA3.1/pKe#83-FLAG vector construct, developed with the anti-FLAG antibody,
Trace 4=Cos cells transfected with the pcDNA3.1 vector (without pKe#83), developed with the anti-FLAG antibody,
Trace 5=FLAG-marked control protein demonstrating the functionality of the anti-FLAG antibody.

The result of this test documents the expression of the pKe#83-FLAG fusion protein in Cos cells, that were transfected with the pcDNA3.1/pKe#83-FLAG vector construct.

### EXAMPLE 4

### Manufacture and Characterization of Antibodies Against the pKe#83 Protein, Along With Immunological Detection of the pKe#83 Protein Via Immunoblot (“Westernblot”), Immune Histology and Enzyme-Linked Immunosorbent Assay (ELISA)

Purified, recombinant pKe#83 non-fusion protein was used for the adjuvant-assisted immunization of rabbits and mice. The details involved in the immunization procedure are generally known in prior art. The rabbits were immunized in response to a customer order placed at Dr. J. Pineda Antikörper-Service (Berlin). Sera were obtained before (“pre-immune serum”) and after (“post-immune serum”) immunization. The IgG fraction was isolated from the sera based on standard procedures by means of ammonium sulfate precipitation. The resulting IgG preparations are referred to as “anti-pKe#83 IgG” below.

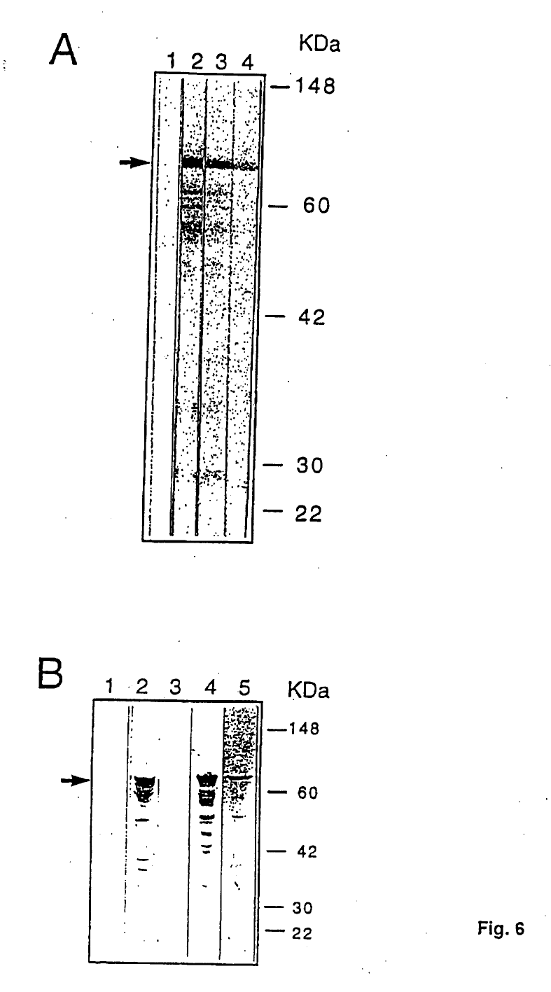

The “anti-pKe#83 IgG” rabbit exhibited a distinct immune reaction with the recombinant pKe#83 protein used for immunization. FIG. 6.A shows the product of this immunoblot procedure. The following applies:
Trace 1=Pre-immune rabbit IgG, 1:10 000 diluted,
Trace 2=anti-pKe#83 IgG 1:50 000 diluted
Trace 3=anti-pKe#83 IgG 1:100 000 diluted
Trace 4=anti-pKe#83 IgG 1:200 000 diluted

The arrow marks the position of the pKe#83 protein.

In addition, the polyclonal rabbit “anti-pKe#83 IgG” and polyclonal mouse “anti-pKe#83 IgG” were used to test cellysates of pKe#83-transfixed Cos cells in an immunoblot procedure for the expression of the pKe#83 protein. FIG. 6.B shows the product of this immunoblot procedure. The following applies:
Trace 1=Pre-immune rabbit IgG,
Trace 2=Rabbit “anti-pKe#83 IgG”,
Trace 3=Normal mouse IgG,
Trace 4=Mouse anti-pKe#83 IgG,
Trace 5=Anti-FLAG antibodies.

Immune histology: A cryotom was used to manufacture 5 μm thick frozen sections of tissues from skin biopsies of clinically unpathological, normal skin and dispase-detached NHEK “sheets” at times T0 and T8. These were air-dried at room temperature and fixed in 100% acetone (100% methanol, 100% ethanol or 4% paraformaldehyde can be used instead of acetone). The sections were then treated according to the “blocking procedure” known in prior art to block non-specific binding sites for the antibody. In this example, two blocking steps are performed: (1) blocking with avidin/biotin and (2) blocking with normal serum. In the first blocking step, the avidin/biotin blocking was performed using the avidin-biotin blocking kit from Vector Laboratories according to the manufacturer's instructions, i.e., incubation was performed at room temperature initially for 15 minutes with the avidin finished solution, and then 15 minutes with the biotin finished solution. Subsequently, the sections were incubated with 10 vol. % normal serum in PBS (normal serum of species from which the second antibody originates, here goat normal serum; PBS=phosphate buffered saline, pH 7.2-7.4) for 15 minutes at room temperature.

After blocking, the sections in PBS were incubated for 1 hour at room temperature with a content of 5 μg/ml rabbit “anti-pKe#83 IgG”. To remove the unbound antibody, the sections were then washed in PBS with a content of 0.2% (weight/volume) bovine serum albumin. This is followed by incubation, for example with a biotin-marked antibody from the goat oriented against rabbit IgG (1:500 diluted in PBS/0.2% BSA; 30 minutes at room temperature), another washing step and the application of a streptavidin marked with the fluorescent dye Cy3 (1:1,000 in PBS/0.2% BSA diluted). A fluorescent dye other than Cy3 can also be used to mark the streptavidin, e.g., FITC. After a last washing step, the sections were covered with a covering agent, e.g., elvanol or histogel, and then analyzed and evaluated under a fluorescence microscope.

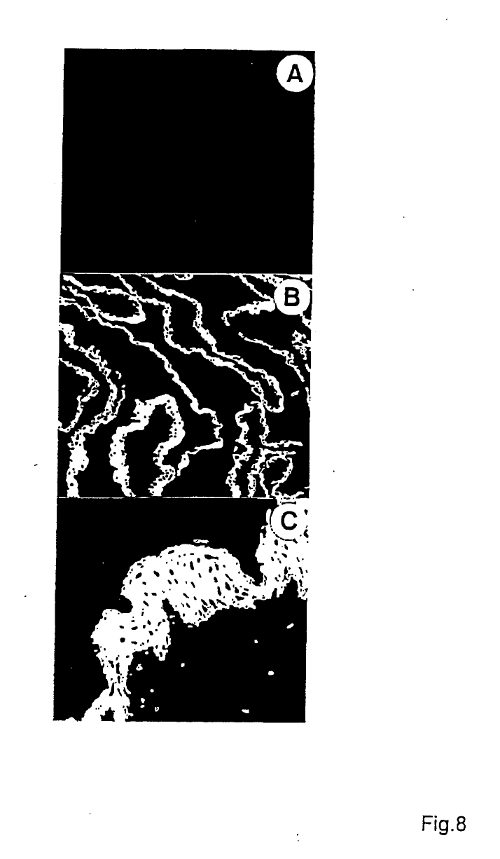

FIG. 8 shows the results obtained from an immune fluorescence detection performed in this manner: The rabbit “anti-pKe#83 IgG” antibody exhibits a weak intracellular and strong cellular membrane-associated immune staining on normal skin sections (FIG. 8.C). The NHEK “sheets” T0 (=immediately after dispase-induced detachment from substrate) exhibit only a slight background staining (FIG. 8.A), while the NHEK “sheets” T8 (=8 hours after dispase-induced detachment from substrate) show a distinct immune staining (FIG. 8.B). This result indicates that little pKe#83 protein was present, or at least detectable, immediately after detachment, but that an elevated expression had already taken place 8, hours later, so that distinctly higher quantities of pKe#83 protein could be detected as a result.

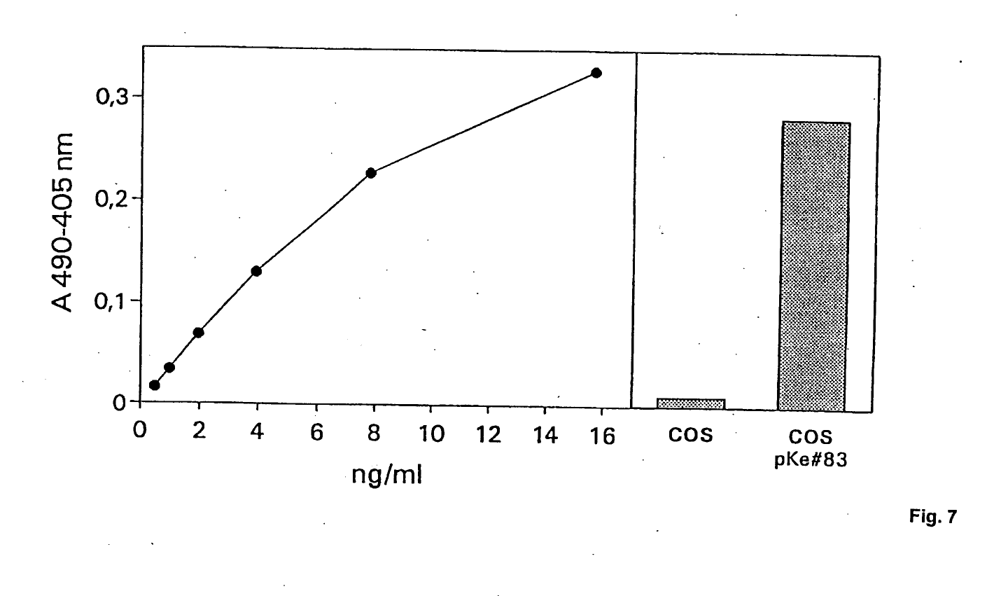

Enzyme-linked immunosorbent assay (ELISA): To quantify the pKe#83 protein in complex solutions, a so-called “sandwich” ELISA (FIG. 7) was performed. To this end, a microtiter plate was coated with an antibody oriented against pKe#83 (e.g., rabbit anti-pKe#83 IgG, 1 μg/well). The still remaining non-specific binding sites of the microtiter plate were then blocked via treatment with 0.1% w/w gelatine in phosphate-buffered saline solution (“PBS/gelatine”). The microtiter plate was subsequently mixed in with various concentrations of the pKe#83 protein as a calibrator, or with dilutions of unknown samples (in which the pKe#83 concentration was to be determined). After a washing step with 0.05% v/v tween-20 in PBS (PBS/tween), the plate was incubated with an IgG preparation from a second species (e.g., with mouse anti-pKe#83 IgG) (e.g., for one hour while shaking at room temperature). After another washing step with PBS/tween, the plate was incubated with a peroxidase-labelled commercial rabbit anti-mouse IgG antibody preparation (e.g., Fc-specific Fab₂-POX from Dianova GmbH, Hamburg). “Peroxidase” here stands for practically any labelling of the antibody, e.g., with enzymes, fluorescence molecules or luminescence molecules. After an additional washing step to remove unbound, enzyme-labelled antibodies, the colorless peroxidase substrate orthophenylene diamine was added, which is converted into a colored product by the peroxidase activity. The color formation is quantified by means of an absorption measurement in a microtiter plate photometer at 490 against 405 nm (ordinate).

FIG. 7 shows the result of such a test. It shows that the color concentration (indicated as absorption in the ordinate) is proportional to the amount of used pKe#83 protein (=“calibrator”, shown in the abscissa). To demonstrate the functionality of the test system, lysates from two different Cos transfectant batches differing in pKe#83 expression were tested at the same time. The Cos cells of the one batch were transfected with the vector construct pcDNA3.1/pKe#83 (“Cos pKe#83” batch), while those of the other batch were transfected with the pcDNA3.1 vector without insert (“Cos” batch).

Cells of these transfectant batches were subjected to lysis according to standard procedures using the Triton X-100 detergent. These lysates were tested in a 1:10 dilution in PBS/tween 20 in the ELISA. Lysates of the “Cos pKe#83” transfectant batch showed a positive reaction. Taking into account the calibrator data, a concentration of approx. 120 ng pKe#83/10⁶ CospKe#83 cells was determined. No pKe#83 protein could be detected in the lysates from the control transfectant “Cos” batches. Consequently, this test procedure can be used to quantify an unknown quantity of the pKe#83 protein in a sample.

The substance orthophenylene diamine here stands for any desired peroxidase substrate that detectably changes its color due to the peroxidase activity. Instead of the polyclonal antibodies used here as an example, use can just as well be made of monoclonal antibodies, which are targeted against the protein pKe#83. Instead of the indirect batch via a marked species-specific anti-IgG antibody, execution can also take place with a directly marked anti-pKe#83 antibody.

### EXAMPLE 5

### Influencing of Keratinocytes With pKe#83-Specific Oligonucleotides

Antisense nucleotides are absorbed by cells, also keratinocytes (compare G. Hartmann et al. 1998: Antisense-Oligonukleotide, Deutsches Ärzteblatt 95, Heft 24, C1115- C1119). They bind in a specific way to the mRNA present in the cell, inhibiting its translation, and hence expression of the corresponding protein (compare Y.-S. Lee, et al. 1997, Definition by specific antisense oligonucleotides of a role for proteinkinase Cα in expression of differentiation markers in normal and neoplastic mouse epidermal keratinocytes, Molecular Carcinogenesis 18, pp. 44-53). Suitable antisense oligonucleotides were manufactured using the pKe#83-specific nucleotide sequence (SEQ ID NO:1 or SEQ ID NO:7). They were set to a concentration of 100 μM with a suitable buffer medium (so-called “oligobuffer”). HaCaT cells were cultivated at 37° C. and 7% CO₂ up to a confluence of 70-80%. The cells were trypsinated off (10 minutes, 0.2% EDTA, 0.1% w/w trypsin, 5-10 minutes,) and set to a concentration of 25,000 cells/ml. 100 μl cell suspension (corresponds to 2,500 cells) were pipetted in per well of a microtiter culture plate (96-well). The cells were incubated for 1 hour, followed by the addition of the antisense oligonucleotide (2 μl of a 100 μM solution) and further incubation for 24-48 hours. The negative control consisted of cell batches to which was added oligonucleotides with the same base distribution, but a randomly selected sequence.

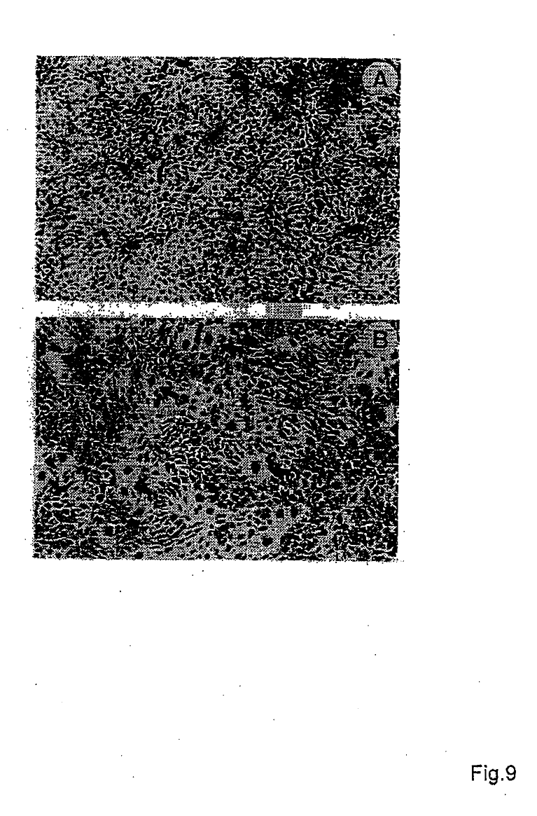

The cells treated in this manner were analyzed under a microscope for phenotypic changes. The result of the microscopic analysis is shown on FIG. 9: FIG. 9.A shows HaCaT cells that were treated with control oligonucleotides, while FIG. 9.B shows HaCaT cells treated with pKe#83-specific antisense oligonucleotides

The microscopic analyses showed that greatly enlarged cells were encountered in the HaCaT cultures treated with antisense oligonucleotides (FIG. 9.B, arrow), which could not be found in the cultures treated with control oligonucleotides. These large cells correspond to differentiated keratinocytes in terms of their morphology. The findings indicate that cells treated with pKe#83-specific antisense oligonucleotides exhibit an increased tendency toward differentiation.

This disclosure further incorporates by reference the electronically submitted text file entitled “Sequence_Listing” created Mar. 13, 2007 and having a size of 40 KB.

1.-22. (canceled)

23. An isolated nucleic acid that specifically hybridizes under stringent conditions to the complement of the sequence set forth in SEQ ID NO: 1, wherein said nucleic acid encodes a polypeptide that is functionally identical to protein pke#83, which is a protein that occurs naturally in human epidermal keratinocytes and which is increasingly expressed when the keratinocytes are in an activated state, as characterized by an elevated expression of the activation markers uPA and uPA-R, said nucleic acid being selected from the group consisting of: (a) SEQ ID NO: 1, and (b) splice variants of SEQ ID NO:

24. The isolated nucleic acid according to claim 23, wherein said nucleic acid is a splice variant of SEQ ID NO: 1 selected from the group consisting of SEQ ID NO: 2 and SEQ ID NO: 5.

25. The isolated nucleic acid according to claim 23, wherein said polypeptide comprises any of SEQ ID NO: 3, SEQ ID NO: 4, or SEQ ID NO: 6.

26. The isolated nucleic acid according to claim 23, wherein said nucleic acid is obtained from a natural, synthetic or semi-synthetic source.

27. The isolated nucleic acid according to claim 23, wherein said nucleic acid is a cDNA.

28. An isolated nucleic acid according to claim 23, wherein said nucleic acid is a sense or antisense oligonucleotide.

29. A recombinant DNA vector comprising a nucleic acid according to claim 23.

30. The recombinant DNA vector molecule according to claim 29, wherein the vector molecule is selected from the group consisting of: the plasmid pUEX-1, the plasmid pGEX-2T and the plasmid pcDNA3. 1.

31. The recombinant DNA vector molecule according to claim 29, wherein the vector molecule is a construct according to the vector protocol on FIG. 2 or the vector protocol on FIG. 3.

32. A transformed host cell comprising the recombinant DNA vector molecule of claim 29.

33. The transformed host cell of 32, wherein the cell has the ability to express protein pKe#83 and wherein the nucleic acid is operably linked to an activatable promoter.

34. The transformed host cell according to claim 32, wherein the promoter is the cytokeratin-14 promoter and the host cell is a keratinocyte, or the promoter is the CMV promoter and the host cell is a cos cell.

35. A method for detecting keratinocytes being in an activated state, as characterized by an elevated expression of the activation markers uPA and uPA-R, said method comprising the steps of:
(i) making a reagent for the detection of the polypetide encoded by the nucleic acid according to claim 23,
(ii) introducing the reagent to keratinocytes, and
(iii) characterizing a greater level of detection of said polypeptide in comparison to control cells as indicative of keratinocytes in an activated state.

36. The method of claim 35, wherein the step of making a reagent further comprises making a reagent that is an isolated antibody that reacts specifically with the polypeptide encoded by the nucleic acid of claim 23.

37. The method of claim 35, further comprising a step of diagnosing dermatological conditions.

38. A method for identifying substances with dermatological applications, comprising the steps of:
(i) introducing the isolated nucleotide of claim 23;
(ii) encoding said polypeptide;
(iii) introducing said polypeptide to said substances;
(iv) monitoring the expression and/or function of said polypeptide;
(v) evaluating said substances for binding said polypeptide; and
(vi) concluding that substances that bind said polypeptide have dermatological applications.

39. The method of 38, wherein said polypeptide is encoded by the amino acid sequence of SEQ ID NO: 3, SEQ ID NO: 4, or SEQ ID NO: 6.

40. The method of 38, further comprising the step of administering a therapeutic amount of said substances that bind said polypeptide.
